(19) United States
(12) Patent Application Publication
(10) Pub. No.: US 2025/0104850 A1
(43) Pub. Date: Mar. 27, 2025

(54) EMERGENCY SUPPORT SYSTEM AND EMERGENCY SUPPORT METHOD

(71) Applicant: Canon Medical Systems Corporation, Otawara-shi (JP)

(72) Inventors: Ryoma HARADA, Tokyo (JP); Yasuyuki MEGURO, Funabashi (JP); Masanobu NAKACHI, Yokohama (JP); Kenta SHIMONISHI, Saitama (JP); Tomoyuki SIBATA, Yokohama (JP)

(73) Assignee: Canon Medical Systems Corporation, Otawara-shi (JP)

(21) Appl. No.: 18/777,192

(22) Filed: Jul. 18, 2024

(30) Foreign Application Priority Data
Sep. 29, 2023 (JP) 2023-169840

(51) Int. Cl. G16H 40/20; G16H 10/60

(52) U.S. Cl. CPC G16H 40/20; G16H 10/60

(57) ABSTRACT

According to one embodiment, an emergency support system includes an emergency transport system configured to support emergency transport and a medical institution system provided in a medical institution that is a destination of transport. The emergency transport system transmits, to the medical institution system to which a patient is to be transported, patient information acquired from an emergency service worker and status confirmation results of the patient including vital information acquired from the emergency service worker. The medical institution system outputs one or more recommended response measures for the patient based on the patient information and the status confirmation results.

### CROSS-REFERENCE TO RELATED APPLICATION

This application is based upon and claims the benefit of priority from prior Japanese Patent Application No. 2023-169840, filed Sep. 29, 2023, the entire contents of which are incorporated herein by reference.

### FIELD

Embodiments described herein relate generally to an emergency support system and an emergency support method.

### BACKGROUND

An emergency support system for performing uniform management, from an emergency call to hospital transfer, of an injured individual has been conventionally known, in which information such as vital signs, findings, and information on the injured individual is input by speech by an emergency service worker, and the input information is provided to a medical institution that is a destination of transport. The information on the injured individual is information such as the age and sex of the injured individual, a situation of the injury, and an estimated injury.

According to the present inventors' study, such an emergency support system, while being adequate for supporting emergency service workers, is considered to leave room for improvement in supporting a medical institution that is a destination of transport.

Specifically, for example, merely providing information input from an emergency service worker is inadequate for appropriately supporting prompt measures and a suitable response at the medical institution that is the destination of transport, which leaves room for improvement.

### BRIEF DESCRIPTION OF THE DRAWINGS

FIG. 1 is a block diagram showing an example of an emergency support system and its peripheral configuration, according to an embodiment.

FIG. 2 is a block diagram showing an example of a configuration of an emergency terminal shown in FIG. 1.

FIG. 3 is a schematic diagram for illustrating a first trained model according to the embodiment.

FIG. 4 is a block diagram showing an example of a configuration of a medical information processing apparatus shown in FIG. 1.

FIG. 5 is a schematic diagram for illustrating a second trained model according to the embodiment.

FIG. 6 is a sequence diagram for illustrating an operation according to the embodiment.

FIG. 7 is a schematic diagram for illustrating a display screen of an emergency terminal shown in FIG. 6.

FIG. 8 is a schematic diagram for illustrating a display screen of an emergency terminal shown in FIG. 6.

FIG. 9 is a schematic diagram for illustrating a display screen of an emergency terminal shown in FIG. 6.

FIG. 10 is a schematic diagram for illustrating a display screen of an emergency terminal shown in FIG. 6.

FIG. 11 is a schematic diagram for illustrating a medical information processing apparatus shown in FIG. 6.

FIG. 12 is a schematic diagram for illustrating a medical information processing apparatus shown in FIG. 6.

FIG. 13 is a schematic diagram for illustrating a medical information processing apparatus shown in FIG. 6.

FIG. 14 is a block diagram showing an example of an emergency support system and its peripheral configuration, according to a modification of the embodiment.

FIG. 15 is a schematic diagram for illustrating a third trained model according to a modification of the embodiment.

### DETAILED DESCRIPTION

In general, according to one embodiment, an emergency support system includes an emergency transport system configured to support emergency transport and a medical institution system provided in a medical institution that is a destination of transport. The emergency transport system includes first processing circuitry. The first processing circuitry is configured to transmit, to the medical institution system to which a patient is to be transported, patient information acquired from an emergency service worker and status confirmation results of the patient including vital information acquired from the emergency service worker. The medical institution system includes second processing circuitry. The second processing circuitry is configured to output one or more recommended response measures for the patient based on the patient information and the status confirmation results.

Hereinafter, an emergency support system and an emergency support method according to an embodiment will be described with reference to the accompanying drawings. Hereinafter, a hospital will be taken as an example of the medical institution; however, the configuration is not limited thereto, and a clinic may be used as the medical institution.

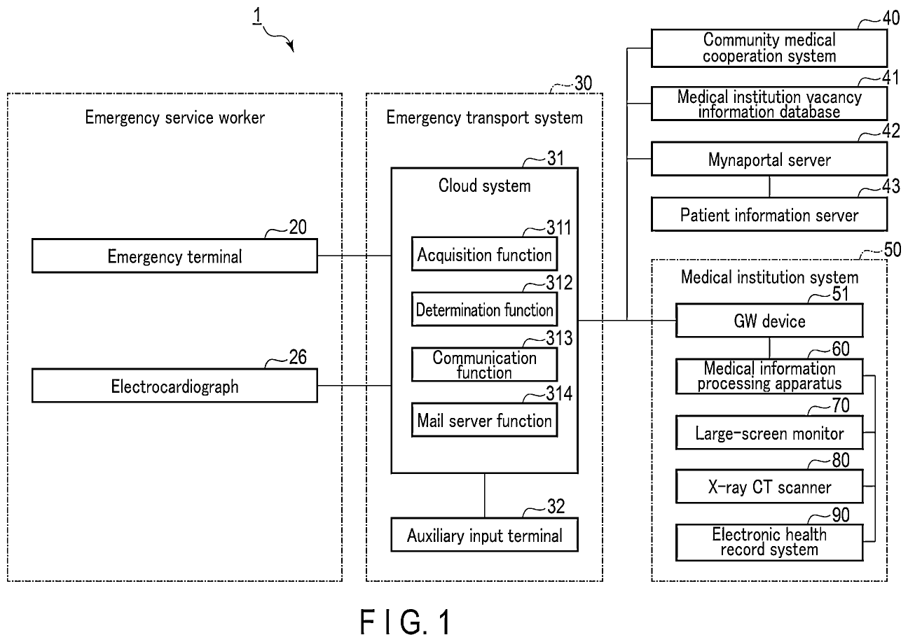

FIG. 1 is a block diagram showing an example of an emergency support system and its peripheral configuration, according to an embodiment. An emergency support system 1 includes an emergency transport system 30 that supports emergency transport, and a medical institution system 50 provided in a medical institution that is a destination of transport. A cloud system 31 of the emergency transport system 30, a community medical cooperation system 40, a medical institution vacancy information database 41, a Mynaportal server 42, and the medical institution system 50 are connected via a network. The Mynaportal server 42 is communicative with a patient information server 43 via a secured network such as an information providing network. On the other hand, an emergency terminal 20 and an electrocardiograph 26 are communicative with the emergency transport system 30. The emergency transport system 30 includes the cloud system 31 and an auxiliary input terminal 32. The medical institution system 50 includes a gateway (GW) device 51, a medical information processing apparatus 60, a large-screen monitor 70, an X-ray CT scanner 80, and an electronic health record system 90. An acquisition function 311, a determination function 312, a communication function 313, and a mail server function 314, which are realized by the cloud system 31, will be described later.

It is assumed that the emergency terminal 20 is an information terminal carried by each of a plurality of emergency service workers, and that each emergency terminal 20 is communicative with the emergency transport system 30.

It is assumed that the electrocardiograph 26 is a device for measuring electrocardiography carried by each emergency service worker, and is capable of obtaining an electrocardiographic waveform of a patient and transmitting electrocardiographic information related to the obtained electrocardiographic waveform to the cloud system 31. Specifically, the electrocardiograph transmits, for example, an email to which a PDF file of the electrocardiographic information is attached to the cloud system 31. The electrocardiographic information includes, for example, an electrocardiographic waveform and analysis results of the electrocardiogram. The analysis results of the electrocardiographic waveform include, for example, measured values such as heart rate, PR interval, QRS duration, QT/QTc, and P-R-T axes, as well as findings regarding abnormalities.

It is assumed that the medical institution system 50 is an intra-hospital system provided in each of a plurality of medical institutions, and is communicative with the emergency transport system 30 and the community medical cooperation system 40.

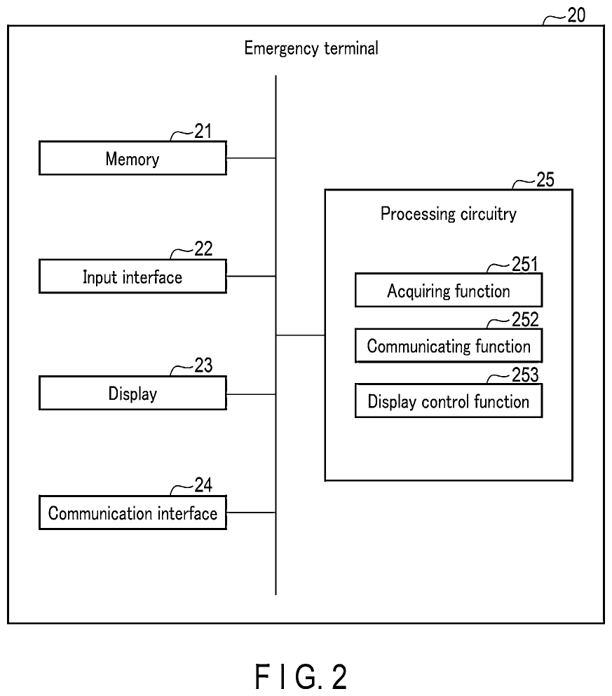

FIG. 2 is a block diagram showing an example of a configuration of the emergency terminal 20 shown in FIG. 1. The emergency terminal 20 includes a memory 21, an input interface 22, a display 23, a communication interface 24, and processing circuitry 25. The memory 21, the input interface 22, the display 23, the communication interface 24, and the processing circuitry 25 are communicatively connected to each other via, for example, a bus.

The memory 21 is, for example, a storage device configured to store various types of information, such as a read-only memory (ROM), a random-access memory (RAM), a hard disk drive (HDD), a solid-state drive (SSD), or an integrated circuit storage device. The memory 21 may be a driving unit or the like configured to read and write various types of information from and into portable storage media such as a CD-ROM drive, a DVD drive, a flash memory, etc. The memory 21 is not necessarily realized by a single storage device. For example, the memory 21 may be realized by a plurality of storage devices. Furthermore, the memory 21 may be placed in another computer connected to the emergency terminal 20.

The memory 21 stores programs and the like to be executed by the processing circuitry 25. Such programs may be, for example, stored in advance in the memory 21. Also, such programs may be stored and distributed in a non-transitory storage medium, and read from the non-transitory storage medium and installed in the memory 21. The memory 21 stores information such as patient information and vital information input from the input interface 22.

The input interface 22 accepts an input operation of various types from the user, and converts the accepted input operation into an electric signal to output it to the processing circuitry 25. The input interface 22 is connected, either physically or via wireless communications of various kinds, to an input device such as a camera, an IC card reader, a microphone, a mouse, a keyboard, a trackball, a switch, a button, a joystick, a touch pad, or a touch panel to which an instruction is input via a touch on its operation surface. The camera is used to photograph the patient and acquire vital information and an injury status. The IC card reader is used to acquire patient information from the patient information card carried by a patient. The microphone is used to input results of observation and consultation of the patient by speech. The input device connected to the input interface 22 may be an input device provided in another computer connected via a network or the like.

The display 23 displays various types of information in accordance with instructions from the processing circuitry 25. For the display 23, any display such as a cathode-ray tube (CRT) display, a liquid crystal display, an organic electroluminescent (EL) display, a light-emitting diode (LED) display, and a plasma display may be suitably employed.

The communication interface 24 performs data communications with an external device such as the cloud system 31.

The processing circuitry 25 is a processor that functions as a core of the emergency terminal 20. The processing circuitry 25 executes a program stored in the memory 21, etc. to realize a function corresponding to the program. The processing circuitry 25 is equipped with, for example, an acquiring function 251, a communicating function 252, and a display control function 253. In the present embodiment, a case will be described where the acquiring function 251, the display control function 253, and the communicating function 252 are realized by a single processor; however, the configuration is not limited thereto. For example, a plurality of independent processors may be combined together to configure processing circuitry such that each of the processors executes a program, thereby realizing the acquiring function 251, the communicating function 252, and the display control function 253.

The acquiring function 251 is a function of acquiring, in response to the emergency service worker's operation, patient information (name, sex, insurance card number, post code/address, weight, height, blood type, infectious disease information, allergy information, contraindicated drug information, disability information, previous disease name(s), consultation history, drug prescription history, etc.), vital information, electrocardiographic information, and results of observation and consultation of the patient. Specifically, the acquiring function 251 stores, into the memory 21, patient information obtained by directly asking questions to the patient through an operation of touching a keyboard, a touchpad, or an operation surface of the input interface 22. The acquiring function 251 performs, for example, verification on an information system called “Mynaportal” using the IC card reader of the input interface 22, acquires patient information from the Mynaportal and stores it into the memory 21. The acquiring function 251 acquires, as vital information, moving image data acquired by photographing the patient from the camera of the input interface 22, and stores it into the memory 21. The acquiring function 251 may analyze the moving image data obtained by photographing the patient to acquire vital data including a respiratory rate or a heart rate of the patient, and store, into the memory 21, vital information including the moving image data and the vital data. The acquiring function 251 acquires results of observation and consultation that have been input by speech from the microphone of the input interface 22, and stores them into the memory 21. The results of observation and consultation are omitted if the patient is unconscious. The electrocardiographic information may be transmitted from the electrocardiograph 26 cloud system 31, without intervention of the emergency terminal 20.

The acquiring function 251 may acquire vital data from a vital measuring device (not illustrated) used by the emergency service worker. The vital data may include a respiratory rate, a blood pressure, a pulse rate (a heart rate), and a body temperature.

The communicating function 252 is a function of performing communications using the communication interface 24, and transmits, in response to the emergency service worker's operation, the patient information, the vital information, and results of observation and consultation in the memory 21 to the cloud system 31 of the emergency transport system 30. The communicating function 252 receives, from the cloud system 31, information such as a transport destination medical institution or candidates for the transport destination medical institution, results of triage, and the like. The communicating function 252 performs, in response to the emergency service worker's operation, communications such as video-format or chat-format conversations with the medical information processing apparatus 60 of the medical institution system 50 via the cloud system 31.

The display control function 253 is a function to display desired information on the display 23 based on the data stored in the memory 21. Specifically, the display control function 253 causes the display 23 to display a display screen including information received from the cloud system 31 or from the medical information processing apparatus 60 via the cloud system 31.

Returning back to FIG. 1, the emergency transport system 30 transmits, to a medical institution system 50 to which the patient is to be transported, the patient information acquired from the emergency terminal 20 of the emergency service worker, and status confirmation results of the patient including the vital information acquired from the emergency terminal 20 and the electrocardiographic information acquired from the electrocardiograph 26. The electrocardiographic information may be included in the vital information. That is, the electrocardiographic information is an example of vital information acquired from the emergency service worker.

The cloud system 31 in the emergency transport system 30 is, for example, a cloud computing system that shares predetermined functions such as the acquisition function 311, the determination function 312, the communication function 313, and the mail server function 314 among a plurality of devices via a network, and realizes them in collaboration. The cloud system 31 may be implemented as, for example, a cloud server including a memory, a communication interface, processing circuitry, etc., which are not illustrated. The cloud server, which is a server constructed in a cloud environment, realizes predetermined functions such as the acquisition function 311, the determination function 312, the communication function 313, the mail server function 314, etc., through execution of programs in the memory by the processing circuitry. The cloud environment refers to an environment that allows the user to access a server via a network such as the Internet.

The acquisition function 311 acquires, from the emergency terminal 20 of the emergency service worker, the vital information and the results of observation and consultation, converts the patient information, the vital information, and the results of observation and consultation into a preset common format, and accumulates them in a memory. The acquisition function 311 acquires a PDF file of the electrocardiographic information attached to an email received by the mail server function 314 from the electrocardiograph 26, and stores it into the memory 21. The acquisition function 311 acquires the patient information from the Mynaportal server 42, and stores it into the memory 21. Also, the acquisition function 311 acquires, from the medical institution vacancy information database 41, medical institution vacancy information, and stores it into the memory 21. If the vital information is moving image data obtained by photographing the patient, the acquisition function 311 analyzes the moving image data to acquire vital data including a respiratory rate or a heart rate of the patient. In accordance therewith, the acquisition function 311 accumulates the vital information including the moving image data and the vital data acquired from the moving image data in the memory. The acquisition function 311 is an example of an acquisition unit.

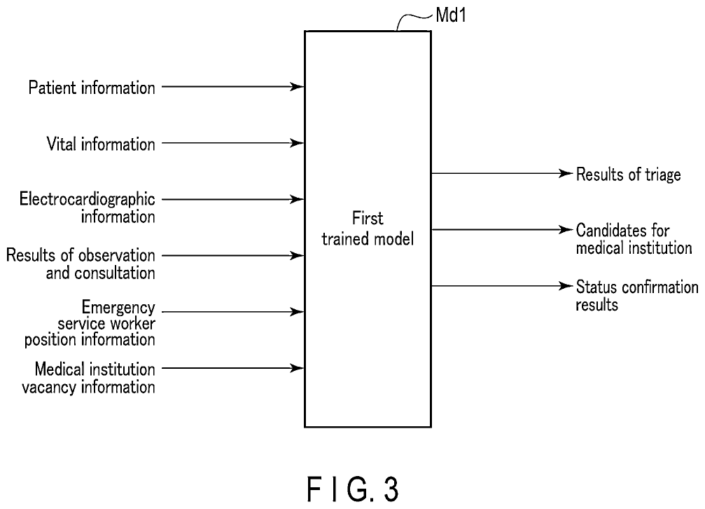

The determination function 312 takes, as an input, the patient information, the vital information, the electrocardiographic information, the results of observation and consultation, the emergency service worker position information, and the medical institution vacancy information to execute a first trained model Md1, thereby outputting results of triage, determination results of candidates for the transport destination medical institution, and status confirmation results of the patient including information on a predicted disease of the patient, as shown in FIG. 3. In this case, the status confirmation results of the patient include the vital information, the electrocardiographic information, and the results of observation and consultation of the patient, as well as the information on the predicted disease of the patient. If the vital information is moving image data, the information on the predicted disease of the patient may be, for example, predicted from an outer appearance of the patient captured in the moving image data. If the electrocardiographic information includes findings related to abnormalities, the information on the predicted disease of the patient may include such findings. The emergency service worker position information can be obtained using, for example, a position information function of the emergency terminal 20. Instead of the candidates for the transport destination medical institution, the first trained model Md1 may output a determination result of the transport destination medical institution. The determination result of the transport destination medical institution refers to, in the case where there is a single candidate for the transport destination medical institution, the medical institution. Examples of the case where there is a single candidate for the transport destination medical institution include a case where there is only one medical institution that has a department capable of responding to emergencies in a predetermined region and a case where there is only one medical institution that has a vacancy among a plurality of medical institutions in a predetermined region. The electrocardiographic information, the input of the emergency service worker position information, and the results of observation and consultation are optional, non-essential additive matters that can be suitably omitted. Also, the determination function 312 transmits, based on a transmission instruction from the emergency terminal 20, the patient information, the status confirmation results of the patient, and the results of triage to the medical information processing apparatus 60 in the medical institution system 50 to which the patient is to be transported. The determination function 312 is an example of a determination unit. The first trained model Md1 is an example of a trained model for transport determination.

The first trained model Md1 is generated by training a model such as a multi-layered network in accordance with a model training program based on training data formed of a set of input data and output data. The input data to the first trained model Md1 includes the patient information, the vital information, the electrocardiographic information, the results of observation and consultation, the emergency service worker position information, and the medical institution vacancy information. The output data from the first trained model Md1 is the results of triage, the candidates for the medical institution, and the status confirmation results. As the machine learning algorithms, a neural network, deep learning, ChatGPT™, Random Forest, etc., are assumed; however, other machine learning algorithms such as support vector machines and kernel regression may also be used. The first trained model Md1 can be regarded as a parameterized composite function in which a plurality of functions are synthesized. The parameterized composite function is defined by a combination of a plurality of adjustable functions and parameters. The first trained model Md1 may be any parameterized composite function that satisfies the above request. The first trained model Md1 is stored in, for example, the memory of the cloud server. If the processing circuitry of the cloud server is configured to execute processing, the first trained model Md1 is read from the memory of the cloud server. The configuration is not limited thereto, and the first trained model Md1 itself may be preset in processing circuitry of the cloud server, as in an ASIC or an FPGA.

Returning back to FIG. 1, the communication function 313 functions as an online meeting system server for providing video-format communications or a chat server for providing communications of chart-format conversations between an emergency terminal 20 of the emergency service worker and the medical information processing apparatus 60 of the medical institution system 50.

The mail server function 314 functions as a mail server for transmitting and receiving emails via the cloud system 31 to and from the emergency terminal 20 and the electrocardiograph 26 of the emergency service worker and the medical information processing apparatus 60 of the medical institution system 50.

The auxiliary input terminal 32 is a terminal that inputs, in response to an operator's operation, medical institution vacancy information to the determination function 312 based on the patient information, the vital information, the electrocardiographic information, and the results of observation and consultation acquired by the acquisition function 311. The auxiliary input terminal 32 may be omitted if the cloud system 31 is configured to access the medical institution vacancy information database 41 and acquire medical institution vacancy information from the medical institution vacancy information database 41.

The community medical cooperation system 40 manages a plurality of items of patient information of each medical institution in a region by associating them with partnership IDs, and transmits, upon receiving a request for patient information from the cloud system 31, a response including the patient information to the requester. The community medical cooperation system 40 may be omitted if the cloud system 31 is configured to access the Mynaportal server 42 and acquire patient information from the patient information server 43 via the Mynaportal server 42.

The medical institution vacancy information database 41 manages a plurality of items of medical institution vacancy information in a region, and transmits, upon receiving a request for medical institution vacancy information from the cloud system 31, a response including the medical institution vacancy information to the cloud system 31. The medical institution vacancy information database 41 may be omitted if the auxiliary input terminal 32 is configured to input medical institution vacancy information to the cloud system 31.

Upon receiving information in a patient's identity document called “My Number Card” from the cloud system 31, the Mynaportal server 42 verifies the received information, and if it is verified to be valid, patient information of the patient is acquired and transmitted to the cloud system 31. A My Number Card is an IC card that stores, into an IC chip, information such as (1) the name, address, date of birth, sex, face image, and an individual number called “My Number” of the patient, which are listed on the card, and (2) an electronic certificate. Of patient information including patient identification information and medical information of the patient, a My Number Card does not store the medical information. Herein, the patient identification information includes name, address, date of birth, and sex. There are two types of electronic certificates: an electronic certificate for signing, and an electronic certificate for user. The electronic certificate for signing is a public-key certificate on which four items of information (name, address, date of birth, and sex) are stored and which allows the four items of information to be read and used. The electronic certificate for user is a public-key certificate used for personal verification. Either may be used as the electronic certificate of the present embodiment; however, an electronic certificate for user is used herein as an example. Upon receiving, for example, an electronic certificate in the My Number Card of the patient from the cloud system 31, the Mynaportal server 42 acquires, if the electronic certificate is verified to be valid, the patient information of the patient from the patient information server 43, and transmits the patient information to the cloud system 31. In this case, the Mynaportal server 42 transmits the electronic certificate to, for example, an ID management server that manages ID information associated with the electronic certificate, transmits a request including the ID information acquired from the ID management server to the patient information server 43, and acquires the patient information from the patient information server 43. The configuration is not limited thereto, and the Mynaportal server 42 can be realized using an existing technique related to Mynaportal.

The patient information server 43 stores the ID information and the patient information related to the electronic certificate of the patient in association with each other, and transmits, in response to a request including the ID information from the Mynaportal server 42, the patient information related to the ID information to the Mynaportal server 42. The configuration is not limited thereto, and the patient information server 43 can be realized using an existing technique that provides information to Mynaportal.

The medical institution system 50 outputs one or more recommended response measures for the patient based on the patient information and the status confirmation results transmitted from the emergency transport system 30.

The GW device 51 in the medical institution system 50 is a device for communicatively connecting the medical information processing apparatus 60 connected to a network in the medical institution and a network outside the medical institution. Hereinafter, the description of intervention of the GW device 51 during communications between the medical information processing apparatus 60 connected to the network in the medical institution and the network outside the medical institution medical institution will be omitted.

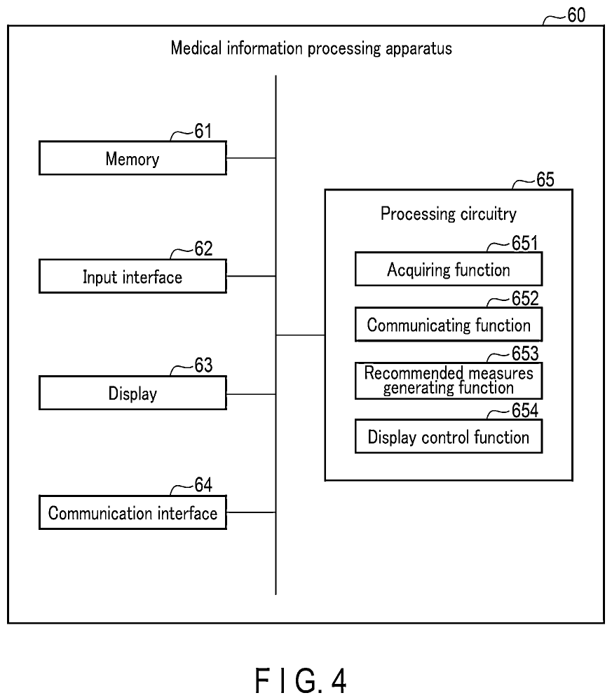

FIG. 4 is a block diagram showing an example of a configuration of the medical information processing apparatus 60 shown in FIG. 1. The medical information processing apparatus 60 includes a memory 61, an input interface 62, a display 63, a communication interface 64, and processing circuitry 65. The memory 61, the input interface 62, the display 63, the communication interface 64, and the processing circuitry 65 are communicatively connected to each other via, for example, a bus.

The memory 61 is, for example, a storage device configured to store various types of information, such as a read-only memory (ROM), a random-access memory (RAM), a hard disk drive (HDD), a solid-state drive (SSD), or an integrated circuit storage device. The memory 61 may be a driving unit or the like configured to read and write various types of information from and into portable storage media such as a CD-ROM drive, a DVD drive, a flash memory, etc. The memory 61 is not necessarily realized by a single storage device. For example, the memory 61 may be realized by a plurality of storage devices. Furthermore, the memory 61 may be placed in another computer connected to the medical information processing apparatus 60.

The memory 61 stores programs, trained models, etc. to be executed by the processing circuitry 65. Such programs and trained models may be, for example, preliminarily stored in the memory 61. Also, such programs and trained models may be stored and distributed in a non-transitory storage medium, and read from the non-transitory storage medium and installed in the memory 61. The memory 61 stores information such as the patient information and the status confirmation results transmitted from the cloud system 31. The memory 61 stores information regarding the patient's electronic health records received from the electronic health record system 90.

The input interface 62 accepts an input operation of various types from the user, and converts the accepted input operation into an electric signal to output it to the processing circuitry 65. The input interface 62 is connected to input devices, for example, a mouse, a keyboard, a trackball, a switch, a button, a joystick, a touch pad, a microphone, or a touch panel to which an instruction is input by a touch on its operation surface. The input device connected to the input interface 62 may be an input device provided in another computer connected via a network or the like.

The display 63 displays various kinds of information such as an integral screen in accordance with an instruction from the processing circuitry 65. For the display 63, any display such as a cathode-ray tube (CRT) display, a liquid crystal display, an organic electroluminescent (EL) display, a light-emitting diode (LED) display, and a plasma display may be suitably employed.

The communication interface 64 performs data communications with the cloud system 31. For example, the communication interface 64 performs data communications with the cloud system 31 in accordance with, for example, a given standard that utilizes a dedicated line. Furthermore, the communication interface 64 performs data communications with the emergency terminal 20 via the cloud system 31 in accordance with a preset existing standard.

The processing circuitry 65 is a processor that functions as a core of the medical information processing apparatus 60. The processing circuitry 65 executes a program stored in the memory 61, etc. to realize a function corresponding to the program. The processing circuitry 65 is equipped with, for example, an acquiring function 651, a communicating function 652, a recommended measures generating function 653, and a display control function 654. In the present embodiment, a case will be described where an acquiring function 651, a communicating function 652, a recommended measures generating function 653, and a display control function 654 are realized by a single processor; however, the configuration is not limited thereto. For example, a plurality of independent processors may be combined together to configure processing circuitry such that the processors execute respective programs to realize the acquiring function 651, the communicating function 652, the recommended measures generating function 653, and the display control function 654.

The acquiring function 651 acquires, from the cloud system 31, information such as the patient information and the status confirmation results, and stores the acquired information into the memory 61. Also, the acquiring function 651 acquires, based on the patient information, information regarding the patient's electronic health record from the electronic health record system 90, and stores the acquired information into the memory 61.

The communicating function 652 performs, in response to a doctor's operation, communications such as video-format or chat-format conversations with the emergency terminal 20 via the cloud system 31.

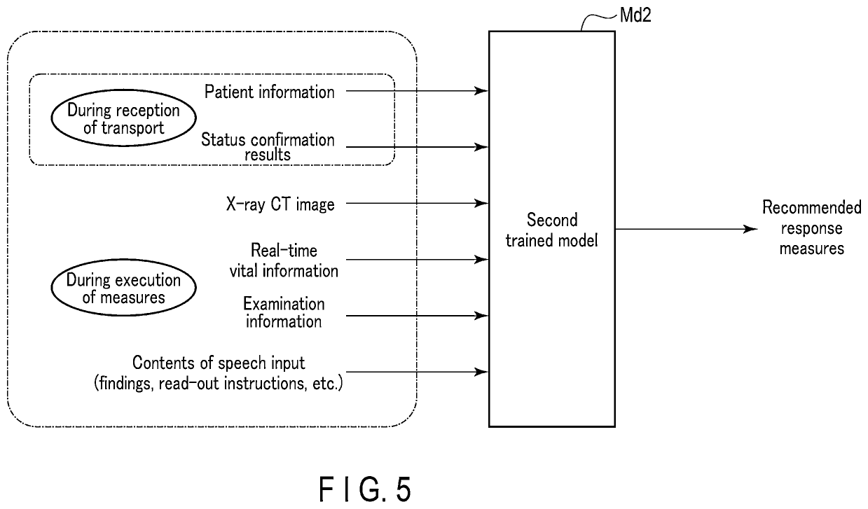

The recommended measures generating function 653 takes, as an input, at least the patient information and the status confirmation results to execute a second trained model Md2, thereby generating the recommended response measures, as shown in FIG. 5. The recommended measures generating function 653 may take, as an input, the patient information, the status confirmation results, examination information of the patient, and vital data collected in real time from the patient to execute the second trained model Md2, thereby updating the recommended response measures. The input data to the second trained model Md2 is not limited thereto, and may include medical images such as X-ray CT images and contents of a speech input made by a doctor (e.g., findings and read-out instructions). The recommended measures generating function 653 is an example of a generating unit.

The second trained model Md2 is generated by training a model such as a multi-layered network in accordance with a model training program based on training data formed of a set of input data and output data. The input data to the second trained model Md2 includes the patient information, the status confirmation results, an X-ray CT image, real-time vital information, examination information, and contents of a speech input (e.g., findings and read-out instructions). The output data from the first trained model Md1 are the recommended response measures in a medical institution. As the machine learning algorithms, a neural network, deep learning, ChatGPT™, Random Forest, etc., are assumed; however, other machine learning algorithms such as support vector machines and kernel regression may also be used. The second trained model Md2 can be regarded as a parameterized composite function in which a plurality of functions are synthesized. The parameterized composite function is defined by a combination of a plurality of adjustable functions and parameters. The second trained model Md2 may be any parameterized composite function that satisfies the above request. The second trained model Md2 is stored in, for example, the memory 61. If the processing circuitry 65 is configured to execute processing, the second trained model Md2 is read from the memory 61. The configuration is not limited thereto, and the second trained model Md2 itself may be preset in the processing circuitry 65, as in an ASIC or an FPGA.

The display control function 654 is a function to display desired information on the display 63 or a large-screen monitor 70 based on the data stored in the memory 61. The display control function 654 causes, for example, the display 63 to display a display screen including the generated recommended response measures. Also, the display control function 654 may cause, for example, the display 63 to display a ranking of a plurality of recommended response measures sequentially obtained by updating of the one or more recommended response measures by the recommended measures generating function 653. The display control function 654 causes the display 63 to display a display screen including information received from the emergency terminal 20, the cloud system 31, or the electronic health record system 90, as well as information generated by the processing circuitry 65. The display control function 654 causes, for example, the display 63 to display a display screen including a screen used for communications such as video-format or chat-format conversations with the emergency terminal 20. Also, real-time vital information, examination information, and X-ray CT images may be displayed. The recommended response measures are not necessarily output in a display format, but may be output in a speech format. The display control function 654 is an example of an output unit that outputs the recommended response measures.

The large-screen monitor 70 is controlled by the medical information processing apparatus 60, and displays a display screen of a size that is at least larger than that of the display 63 of the medical information processing apparatus 60.

The X-ray CT scanner 80 is a medical image capturing apparatus that captures a CT image of the patient. The X-ray CT scanner 80 is capable of transmitting the captured CT image to the medical information processing apparatus 60.

The electronic health record system 90 is an apparatus for storing information regarding electronic health records of patients.

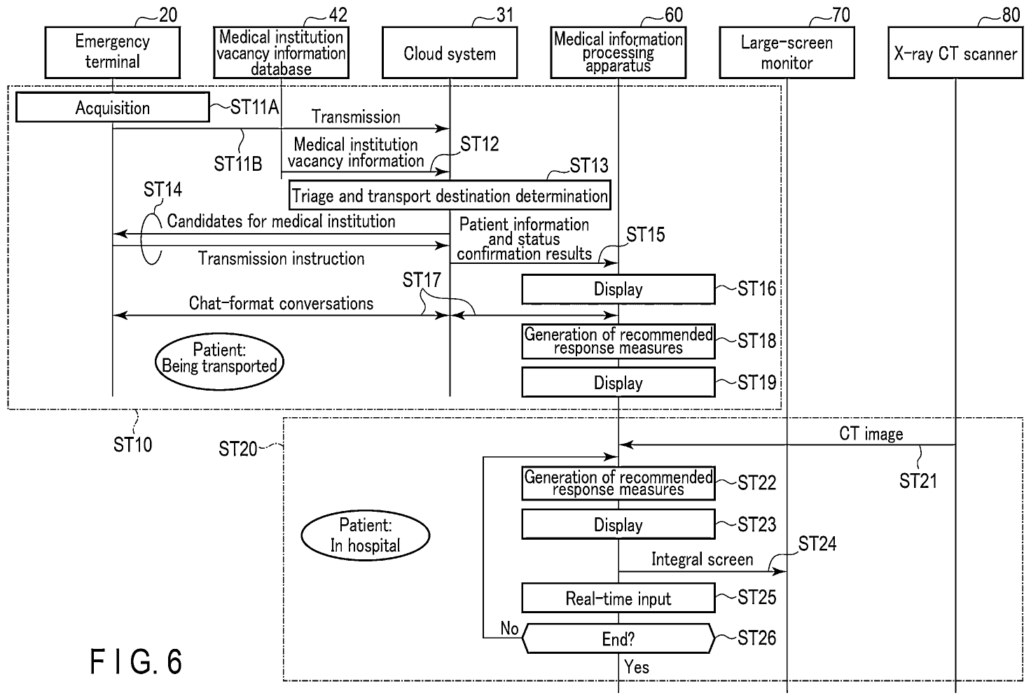

Next, the operation of the emergency support system 1 with the above-described configuration will be described with reference to FIGS. 6-13. In the following description, the operation will be divided into step ST10 showing an operation during emergency transport of a patient and step ST20 showing an operation while the transported patient is in a hospital, as shown in FIG. 6. Step ST10 includes steps ST11A to ST19.

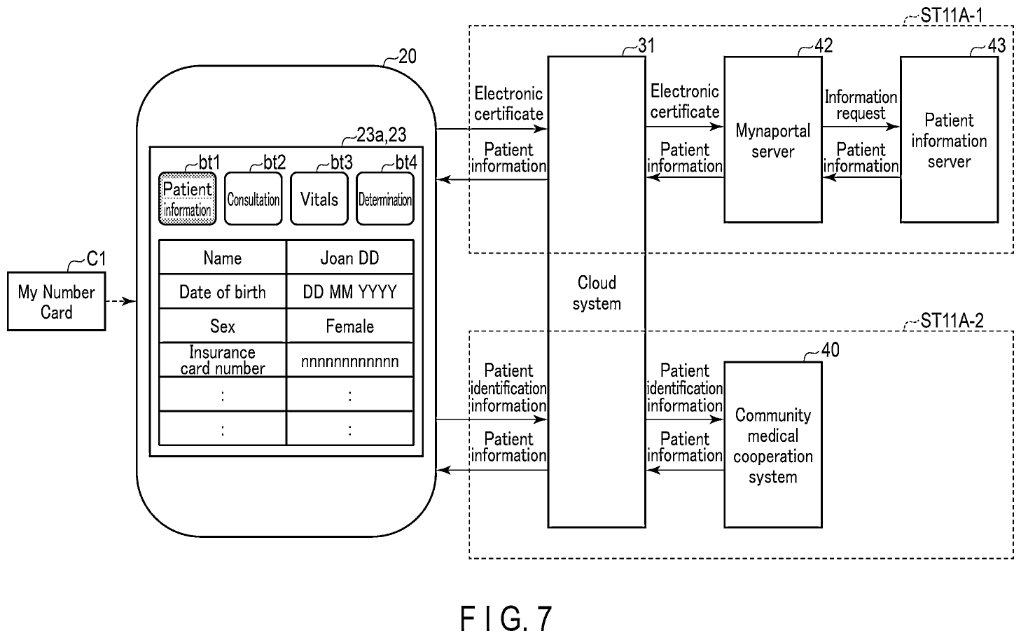

First, at step ST11A, the processing circuitry 25 of the emergency terminal 20 acquires patient information in response to an emergency service worker's operation on the input interface 22. For example, the emergency terminal 20 reads a My Number Card C1 carried by the patient with an IC card reader (not illustrated) of the input interface 22 to perform verification on Mynaportal, and stores patient information in Mynaportal into the memory 21. In this case, the emergency terminal 20 transmits an electronic certificate read from the My Number Card C1 to the Mynaportal server 42 via the cloud system 31, as shown by, for example, step ST11A-1 in FIG. 7. The Mynaportal server 42 verifies the electronic certificate, and requests, if it has been verified to be valid, the patient information server 43 of patient information and transmits the obtained patient information to the emergency terminal 20 via the cloud system 31. The emergency terminal 20 stores the received patient information to the memory 21. The configuration is not limited thereto, and the emergency terminal 20 may be configured to transmit patient identification information read from the My Number Card C1, such as the name, the address, the date of birth, and the sex of the patient, to the community medical cooperation system 40 via the cloud system 31, as shown by, for example, step ST11A-2 in FIG. 7. In this case, the community medical cooperation system 40 transmits patient information stored in association with patient identification information to the emergency terminal 20 via the cloud system 31. The emergency terminal 20 stores the received patient information to the memory 21. Alternatively, the emergency terminal 20 may read patient information from an IC card (not illustrated) storing the patient information, instead of the My Number Card C1, and store the read patient information in the memory 21. Alternatively, patient information obtained by directly asking questions to the patient may be stored in the memory 21. Thereafter, in either case, the processing circuitry 25 causes the display 23 to display a display screen 23a including patient information in the memory 21. Examples of the patient information that may be suitably used include name, date of birth, sex, insurance card number, post code/address, weight, height, blood type, infectious disease information, allergy information, contraindicated drug information, disability information, previous disease name(s), consultation history, drug prescription history, etc. Of the above-listed items of the patient information, the name, the date of birth, the sex, and the address correspond to the patient identification information that can be read from the My Number Card C1. Of the above-listed items of the patient information, the information such as the insurance card number, the weight, the height, the blood type, the infectious disease information, the allergy information, the contraindicated drug information, the disability information, the previous disease name(s), the consultation history, and the drug prescription history correspond to medical information that is not stored in the My Number Card C1. The display screen 23a includes a first button bt1 for executing an operation mode related to the patient information, a second button bt2 for executing an operation mode related to consultation, a third button bt3 for executing an operation mode related to vital information, and a fourth button bt4 for executing an operation mode related to determination of the transport destination.

The processing circuitry 25 of the emergency terminal 20 stores, in the memory 21, an operation of the second button bt2 and text data indicating results of observation and consultation, in response to a speech input made by the emergency service worker. If the patient is unconscious, a situation of the scene of an accident, for example, may be input either by speech or manually as the results of observation and consultation.

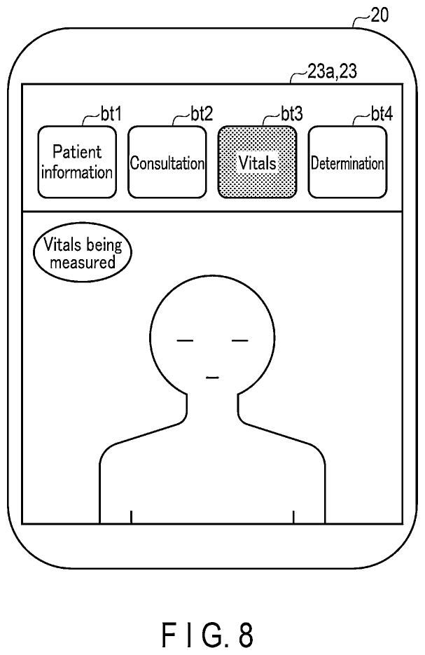

In response to the emergency service worker's operation, the processing circuitry 25 of the emergency terminal 20 stores, in the memory 21, an operation of the third button bt3 and moving image data obtained by photographing the patient as vital information, as shown in FIG. 8. If the patient is unconscious, a situation of the scene of an accident, for example, may be input by speech as the results of observation and consultation. In this manner, the emergency terminal 20 acquires the patient information, the vital information, and the results of observation and consultation of the patient.

Subsequently, at step ST11B, the emergency terminal 20 transmits, in response to the emergency service worker's operation, the patient information, the vital information, and the results of observation and consultation to the cloud system 31. Also, in parallel with steps ST11A and ST11B, the electrocardiograph 26 measures, in response to the emergency service worker's operation, an electrocardiogram of the patient, and transmits an email to which electrocardiographic information related to the electrocardiographic waveform is attached to the cloud system 31. It suffices that the transmission of an email to which the electrocardiographic information is attached is executed prior to step ST13, and such transmission is not necessarily executed in parallel with steps ST11A and ST11B. For example, an email to which electrocardiographic information is attached may be transmitted between step ST12 and step ST13.

At step ST12, the auxiliary input terminal 32 transmits, in response to an operator's operation, medical institution vacancy information to the cloud system 31.

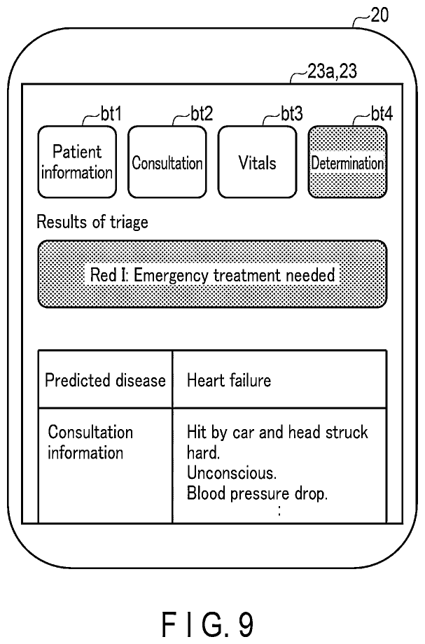

At step ST13, the cloud system 31 performs triage and determination of candidates for the transport destination based on the patient information, the vital information, the electrocardiographic information, the results of observation and consultation, the emergency service worker position information, and the medical institution vacancy information. For example, the cloud system 31 takes, as an input, the acquired patient information, the vital information, the electrocardiographic information, and the results of observation and consultation, the emergency service worker position information, and the medical institution vacancy information to execute a first trained model Md1, thereby outputting results of triage, determination results of candidates for the transport destination medical institution, and status confirmation results including information on a predicted disease of the patient. Thereby, in the emergency terminal 20, a display screen 23a including the results of triage and the information on the predicted disease are displayed in response to the emergency service worker's operation, as shown in FIG. 9.

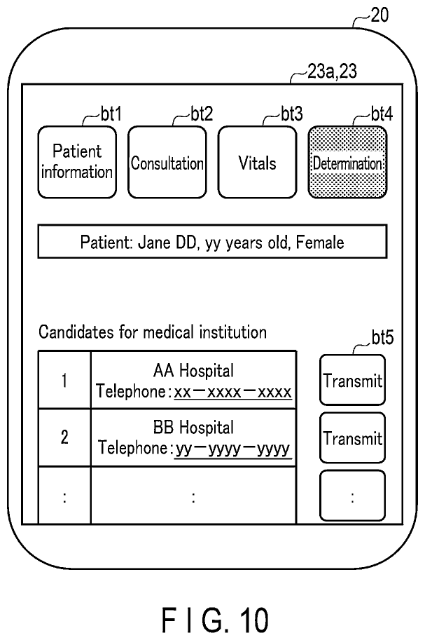

At step ST14, in the emergency terminal 20, as shown in FIG. 10, a display screen 23a including a list of candidates for the transport destination medical institution, a fifth button bt5 for making a notification of a transmission instruction to the selected transport destination, and information on a link to a telephone number for performing video-format or chat-format conversations with the selected candidate is displayed in response to the emergency service worker's operation. In the emergency terminal 20, in response to an operation on the fifth button bt5, a transmission instruction to the transport destination medical institution is transmitted to the cloud system 31.

At step ST15, the cloud system 31 transmits, based on the transmission instruction, the patient information, the status confirmation results of the patient, and the results of triage to the medical information processing apparatus 60 in the medical institution system 50 to which the patient is to be transported.

At step ST16, upon receiving the patient information, the patient status confirmation results, and the results of triage, the processing circuitry 65 in the medical information processing apparatus 60 stores the patient information, the patient status confirmation results, and the results of triage into the memory 61. Also, the processing circuitry 65 causes the display 63 to display the patient information, the patient status confirmation results, and the results of triage in the memory 61.

At step ST17, in response to the emergency service worker's operation, the emergency terminal 20 executes communications with the cloud system 31 to perform video-format or chat-format conversations with the medical information processing apparatus 60. Thereby, video-format or chat-format conversations are performed via the cloud system 31 between the emergency terminal 20 operated by the emergency service worker and the medical information processing apparatus 60 operated by a doctor.

At step ST18, in response to the doctor's operation, the processing circuitry 65 of the medical information processing apparatus 60 takes, as an input, at least the patient information and the status confirmation results to execute a second trained model Md2, thereby generating one or more recommended response measures.

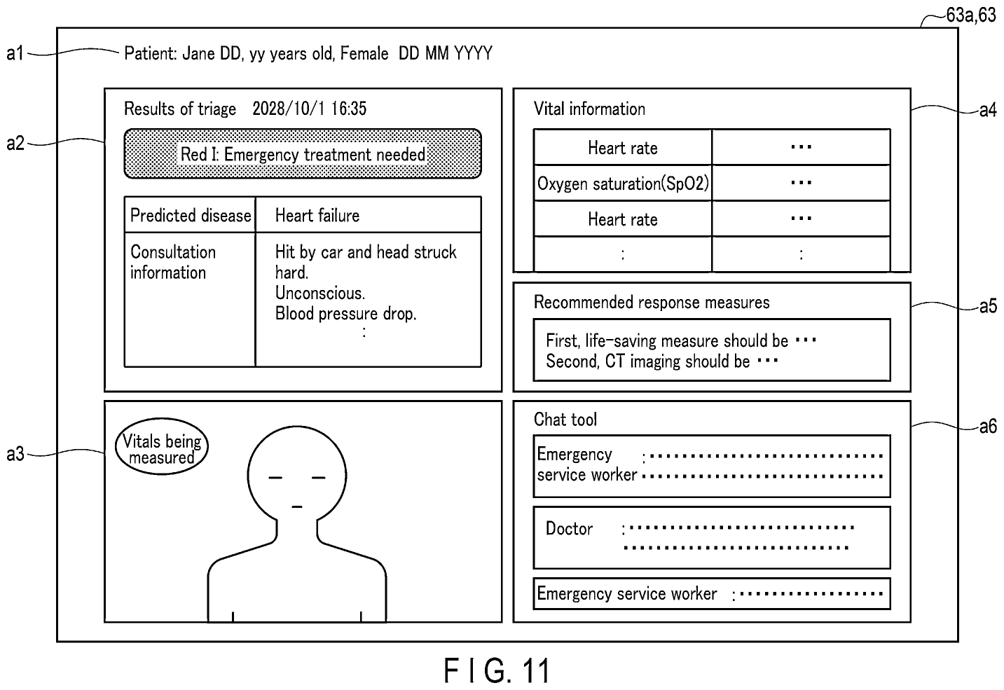

At step ST19, the processing circuitry 65 of the medical information processing apparatus 60 causes the display 63 to display a display screen including the generated recommended response measures. The processing circuitry 65 causes an integral screen 63a in which a first region a1, a second region a2, a third region a3, a fourth region a4, a fifth region a5, and a sixth region a6 are integrated as a display screen, as shown in FIG. 11, for example. The first region a1 contains the patient information. The second region a2 contains the results of triage, the information on the predicted disease, and the results of observation and consultation. The third region a3 contains, of the vital information, moving image data. The third region a3 may contain the electrocardiographic information, in place of the moving image data. The fourth region a4 contains, of the vital information, vital data other than the moving image data. The fifth region a5 contains the recommended response measures. The sixth region a6 contains records of video-format or chat-format conversations. With the foregoing, step ST10 including steps ST11 to ST19 comes to an end.

Upon emergency transport of the patient to the medical institution after the end of step ST10, step ST20, which is an operation performed while the patient is in the hospital, is started. Step ST20 includes steps ST21 to ST26.

At step ST21, in response to an operation on the X-ray CT scanner 80 by a doctor, the X-ray CT scanner 80 performs CT imaging of the patient. Thereafter, the medical information processing apparatus 60 receives a CT image of the patient from the X-ray CT scanner 80.

At step ST22, upon receiving the CT image of the patient, the processing circuitry 65 of the medical information processing apparatus 60 takes, as an input, the patient information, the status confirmation results, and the CT image to execute a second trained model Md2, thereby generating (updating) the one or more recommended response measures. If the doctor who has confirmed the CT image performs a speech input, the processing circuitry 65 executes the second trained model Md2 by including the contents of the speech input in the input data, thereby updating the recommended response measures. In this case, it is preferable that an interactive AI such as ChatGPT™ be used as the second trained model Md2. At step ST22, every time information to be taken as an input to the second trained model Md2 is received, the processing circuitry 65 executes the second trained model Md2, thereby generating (updating) the one or more recommended response measures.

At step ST23, the processing circuitry 65 of the medical information processing apparatus 60 causes the display 63 to display an integral screen 63a including the generated recommended response measures.

At step ST24, the processing circuitry 65 of the medical information processing apparatus 60 causes the large-screen monitor 70 to display an integral screen similar to the integral screen 63a at step ST23. Thereby, the doctor performs measures on the patient, while confirming the recommended response measures on the large-screen monitor 70.

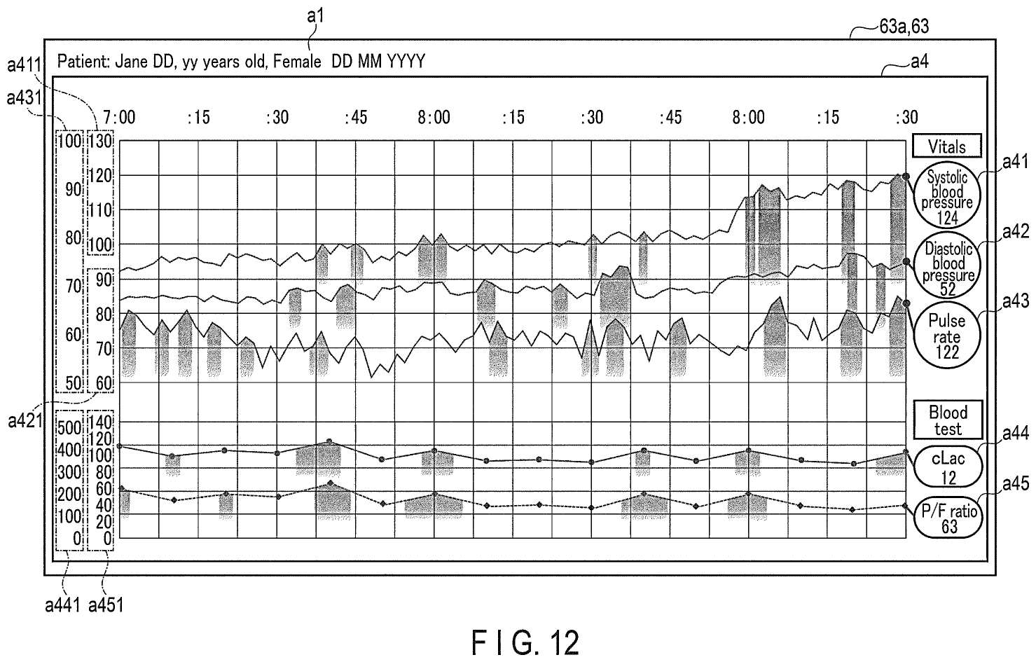

At step ST25, vital data collected in real time from the patient is input to the medical information processing apparatus 60. The processing circuitry 65 of the medical information processing apparatus 60 stores the vital data in the memory 61. Also, in response to the doctor's operation, the processing circuitry 65 causes the display 63 to display an integral screen 63a in which a first region a1 containing the patient information and a fourth region a4 containing time-series vital data are integrated, as shown in FIG. 12. In FIG. 12, the vital data includes, for example, a systolic blood pressure a41, a diastolic blood pressure a42, a pulse rate a43, or vital data to be displayed in real time. In FIG. 12, examination information of a blood test includes, for example, a cLac a44 and a P/F ratio a45, or a desired item of examination information to be displayed. On the vertical axis of the graph in the fourth region, a value a411 of the systolic blood pressure a41, a value a421 of the diastolic blood pressure a42, a value a431 of the pulse rate a43, a value a441 of cLac a44, and a value a451 of the P/F ratio a45 are shown. The vertical axis of the graph represents time.

At step ST26, the processing circuitry 65 of the medical information processing apparatus 60 determines, according to whether or not an end instruction has been input, whether or not to end the processing. If the processing is not to be ended, the processing returns to step ST22, and repeatedly executes processing from step ST22 to step ST26.

At repeatedly executed step ST22, the processing circuitry 65 takes, as an input, the patient information, the status confirmation results, the examination information of the patient, and the vital data collected in real time from the patient to execute the second trained model Md2, thereby updating the one or more recommended response measures.

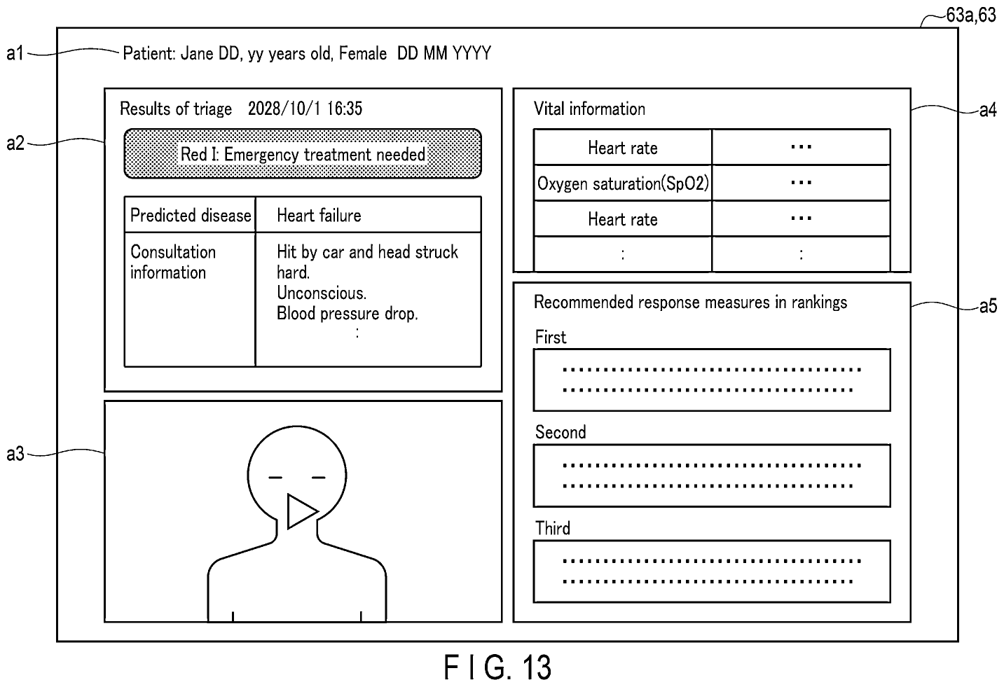

At repeatedly executed step ST23, the processing circuitry 65 causes the display 63 to display a ranking of a plurality of recommended response measures sequentially obtained by updating the one or more recommended response measures, as shown in the fifth region a5 of FIG. 13. A notification of the recommended response measures is made by speech.

On the other hand, if an end instruction is input at step ST26, the processing circuitry 65 ends the processing.

As described above, according to the embodiment, an emergency support system 1 includes an emergency transport system 30 that supports emergency transport, and a medical institution system 50 provided in a medical institution that is a transport destination. The emergency transport system 30 transmits, to a medical institution system 50 to which the patient is to be transported, patient information acquired from an emergency service worker and status confirmation results of the patient including vital information acquired from the emergency service worker. The medical institution system 50 outputs recommended response measures for the patient based on the patient information and the status confirmation results. It is thereby possible to provide prompt measures and a suitable response for supporting the transport destination medical institution. That is, according to the embodiment, unlike the conventional triage technique in which the transport destination is simply determined, it is possible to integrate the emergency transport system 30 into the medical institution system 50, and to present recommended response measures in the medical institution system 50, thus enabling a smooth response on the scene of emergency where every moment counts.

Moreover, according to the embodiment, the medical information processing apparatus 60 of the medical institution system 50 takes, as an input, at least the patient information and the status confirmation results to execute a second trained model Md2, thereby generating one or more recommended response measures. Also, the medical information processing apparatus 60 outputs the recommended response measures. It is thereby possible, in addition to the above-described effect, to obtain recommended response measures using a trained model.

Furthermore, according to the embodiment, the medical information processing apparatus 60 takes, as an input, patient information, status confirmation results, examination information of the patient, and vital data collected in real time from the patient to execute a second trained model Md2, thereby updating the one or more recommended response measures. It is thereby possible, in addition to the above-described effect, to update the recommended response measures according to a real-time situation of the patient.

Moreover, according to the embodiment, the medical information processing apparatus 60 causes the display 63 to display a ranking of a plurality of recommended response measures sequentially obtained by updating of the one or more recommended response measures. Thereby, recommended response measures prior to the updating are displayed in lower ranks, and recommended response measures after the update are displayed in higher ranks. Accordingly, since the recommended response measures are erased after being moved to the bottom of the screen rank by rank, it is possible to suppress confusion that may be caused by sudden update (erasure) of the recommended response measures being used.

Furthermore, according to the embodiment, the cloud system 31 in the emergency transport system 30 takes, as an input, patient information and vital information as well as medical institution vacancy information to execute a first trained model Md1, thereby outputting transport destination determination results or transport destination candidate determination results, and status confirmation results including information on a predicted disease of the patient. It is thereby possible, in addition to the above-described effect, to provide, to an emergency service worker, the transport destination determination results or the transport destination candidate determination results, and the status confirmation results including the information on the predicted disease of the patient.

Moreover, according to the embodiment, the cloud system 31 in the emergency transport system 30 takes, as an input, patient information, vital information, emergency service worker position information, and medical institution vacancy information to execute a first trained model Md1. With the configuration of further inputting the emergency service worker position information, it is possible, in addition to the above-described effect, to obtain transport destination determination results or transport destination candidate determination results in consideration of the position of the emergency service worker. Thereby, it can be expected, for example, to determine a medical institution close to the position of the emergency service worker as a transport destination or a candidate therefor, and to exclude a medical institution far from the position of the emergency service worker from a transport destination or a candidate therefor.

Furthermore, according to the embodiment, the cloud system 31 executes a first trained model Md1 to further output results of triage of the patient. It is thereby possible, in addition to the above-described effect, to provide, to an emergency service worker, the results of triage of the patient. To be more specific, an AI-based triage technique, which performed triage using artificial intelligence (AI) during the COVID-19 pandemic, has conventionally been known. While not being particularly problematic, the AI-based triage technique is applicable only to those infected with COVID-19, and performs determinations without considering cases other than COVID-19. In addition, the AI-based triage technique requires diagnosis results (clinical data) of a patient already transported to an institution and diagnosed by a doctor, which makes it difficult to promptly and suitably identify a transport destination. On the other hand, the first trained model Md1 of the present embodiment is not limited to those infected with COVID-19, and is capable of outputting results of triage while eliminating the need for the results of diagnosis made by the doctor.

Moreover, according to the embodiment, the vital information may be moving image data obtained by photographing the patient. The cloud system 31 of the emergency transport system 30 analyzes the moving image data to acquire vital data including a respiratory rate or a heart rate of the patient. In this case, it is possible, in addition to the above-described effect, to obtain the patient's vital data simply by an emergency service worker capturing a moving image of the patient. Also, by acquiring vital data from the moving image data, it is possible to simplify obtainment of the vital data.

Furthermore, according to the embodiment, the vital information may be vital data including a respiratory rate or a heart rate obtained by analyzing moving image data obtained by photographing the patient. In this case, it is possible, in addition to the above-described effect, to acquire vital data by an emergency service worker analyzing the moving image data, and to acquire the patient's vital data from the emergency service worker without the cloud system 31 performing moving image analysis.

### Modification 1

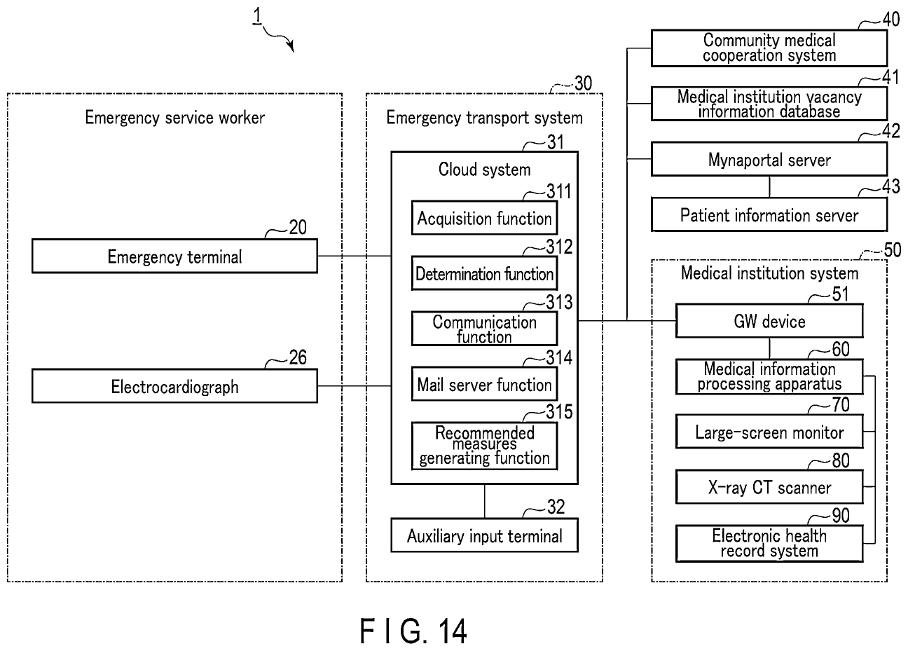

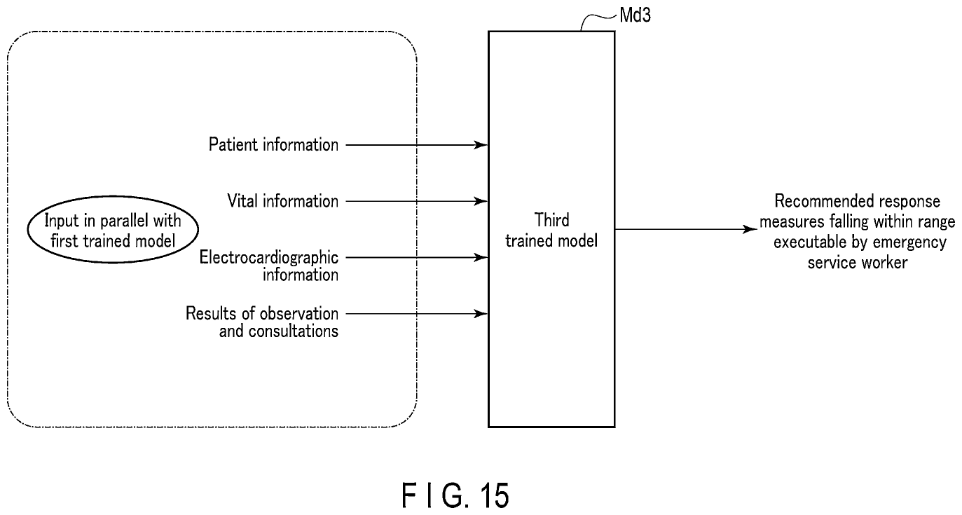

In the embodiment, the cloud system 31 does not generate recommended response measures; however, the configuration is not limited thereto. For example, the cloud system 31 may also execute a recommended measures generating function 315, as shown in FIG. 14. In this case, the recommended measures generating function 653 of the medical information processing apparatus 60 is not omitted, and is executed as described above. The recommended measures generating function 315 of the cloud system 31 takes, as an input, at least patient information and vital information to execute a third trained model Md3, thereby generating recommended response measures that fall within a range executable by an emergency service worker, as shown in FIG. 15. In the example of FIG. 15, the input data includes patient information, vital information, electrocardiographic information, and results of observation and consultations, and is input to the third trained model Md3 in parallel with the first trained model Md1. The recommended measures generating function 315 outputs, to the emergency terminal 20, the generated recommended response measures that fall within the range executable by an emergency service worker. The third trained model Md3 can be realized by a technique similar to the above-described technique for realizing the second trained model Md2, except for the contents of the input data and the output data. The recommended measures generating function 315 is an example of a generating unit for emergency service workers and an output unit for emergency service workers. The third trained model Md3 is an example of a trained model for emergency service workers. According to Modification 1 described above, it is possible, in addition to the effects of the embodiment, to support emergency service workers with response measures by outputting recommended response measures to the emergency terminal 20.

### Modification 2

In the embodiment and Modification 1, the first trained model Md1, the second trained model Md2, and the third trained model Md3 have been used; however, the configuration is not limited thereto. For example, the first trained model Md1, the second trained model Md2, and the third trained model Md3 may be respectively replaced with predetermined logic determinations. According to Modification 2 described above, it is possible, in addition to the effects of the embodiment, to reduce the burden of machine learning for constructing a trained model.

With at least one embodiment described above, it is possible to provide prompt measures and a suitable response for supporting the transport destination medical institution.

The term “processor” used in the descriptions of the embodiments means, for example, a central processing unit (CPU), a graphics processing unit (GPU), or a circuit such as an application-specific integrated circuit (ASIC), a programmable logic device (e.g., simple programmable logic device (SPLD), and a complex programmable logic device (CPLD), and a field programmable gate array (FPGA)). The processor reads and executes a program stored in a memory to realize a function. If the processor is, for example, a CPU, the processor reads and executes a program stored in a storage circuit to realize a function. On the other hand, if the processor is, for example, an ASIC, the function is directly incorporated as a logic circuit in the circuit of the processor, instead of the program being stored in the storage circuit. Each processor in the present embodiment is not limited to a single circuitry-type processor, and multiple independent circuits may be combined and integrated as a single processor to realize the intended functions. Furthermore, a plurality of structural components shown in any one of FIGS. 1, 2, 4 and 14 may be integrated into a single processor to realize the functions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An emergency support system comprising an emergency transport system configured to support emergency transport and a medical institution system provided in a medical institution that is a destination of transport, wherein
the emergency transport system includes first processing circuitry configured to transmit, to the medical institution system to which a patient is to be transported, patient information acquired from an emergency service worker and status confirmation results of the patient including vital information acquired from the emergency service worker, and
the medical institution system includes second processing circuitry configured to output one or more recommended response measures for the patient based on the patient information and the status confirmation results.

2. The emergency support system according to claim 1, wherein
the second processing circuitry is configured to generate the one or more recommended response measures by taking, as an input, at least the patient information and the status confirmation results to execute a trained model;
and output the one or more recommended response measures.

3. The emergency support system according to claim 2, wherein
the second processing circuitry is configured to update the one or more recommended response measures by taking, as an input, the patient information, the status confirmation results, examination information of the patient, and vital data collected in real time from the patient to execute the trained model.

4. The emergency support system according to claim 3, wherein
the second processing circuitry causes the display to display a ranking of a plurality of recommended response measures sequentially obtained by the updating of the one or more recommended response measures.

5. The emergency support system according to claim 1, wherein
the first processing circuitry is configured to output transport destination determination results or transport destination candidate determination results, and status confirmation results including information on a predicted disease of the patient by taking, as an input, the patient information, the vital information, and medical institution vacancy information to execute a trained model for transport determination.

6. The emergency support system according to claim 5, wherein
the first processing circuitry configured to take, as an input, the patient information, the vital information, position information of the emergency service worker, and the medical institution vacancy information to execute the trained model for transport determination.

7. The emergency support system according to claim 5, wherein
the first processing circuitry is further configured to output results of triage of the patient by executing the trained model for transport determination.

8. The emergency support system according to claim 1, wherein
the vital information is moving image data obtained by photographing the patient, and
the first processing circuitry is configured to acquire vital data including a respiratory rate or a heart rate of the patient by analyzing the moving image data.

9. The emergency support system according to claim 1, wherein
the vital information is vital data including a respiratory rate or a heart rate obtained by analyzing moving image data obtained by photographing the patient.

10. The emergency support system according to claim 1, wherein
the first processing circuitry is configured to generate recommended response measures falling within a range executable by the emergency service worker by taking, as an input, at least the patient information and the vital information to execute a trained model for the emergency service worker; and output the recommended response measures falling within the range executable by the emergency service worker.

11. An emergency support method executed by an emergency transport system configured to support emergency transport and a medical institution system provided in a medical institution that is a destination of transport, wherein
the emergency transport system transmits, to the medical institution system to which a patient is to be transported, patient information acquired from an emergency service worker and status confirmation results of the patient including vital information acquired from the emergency service worker, and
the medical institution system outputs one or more recommended response measures for the patient based on the patient information and the status confirmation results.

12. The emergency support method according to claim 11, further comprising:
acquiring, from the emergency service worker, electrocardiographic information related to an electrocardiographic waveform measured from the patient by the emergency transport system, prior to the transmitting, wherein
the status confirmation results further include electrocardiographic information acquired from the emergency service worker.
